(19) United States
(12) Patent Application Publication
(10) Pub. No.: US 2026/0199645 A1
(43) Pub. Date: Jul. 16, 2026

(54) ADJUSTABLE SHUNTING SYSTEMS WITH SHAPE MEMORY ACTUATORS AND ASSOCIATED SYSTEMS AND METHODS

(71) Applicant: Shifamed Holdings, LLC, Campbell, CA (US)

(72) Inventors: Miles Alexander, Fremont, CA (US); Isabel Mulvihill, San Francisco, CA (US); Ian Baranowski, Menlo Park, CA (US); Brian Fahey, Menlo Park, CA (US)

(21) Appl. No.: 19/136,855

(22) PCT Filed: Dec. 20, 2023
(86) PCT No.: PCT/US2023/085189

§ 371 Date: Jun. 6, 2025

Related U.S. Application Data
(60) Provisional application No. 63/477,104, filed on Dec. 23, 2022.
(60) Provisional application No. 63/511,132, filed on Jun. 29, 2023.

(51) Int. Cl. A61M 27/00

(52) U.S. Cl. CPC A61M 27/002; A61M 2205/0266; A61M 2205/04; A61M 2205/3331

(57) ABSTRACT

The present technology provides adjustable shunting systems with actuation assemblies that can be selectively adjusted to change a level of therapy provided by the shunt. The actuation assemblies can include a shape memory actuator having a plurality of leaflets or projections forming a conical shape with openings on both ends of the cone. The actuation assemblies can further include one or more membranes that individually jacket or cover individual projections of the plurality of projections to form a lumen extending through the conical shape.

### CROSS-REFERENCE TO RELATED APPLICATION(S)

This application claims the benefit of U.S. Provisional Patent Application No. 63/477,104, filed Dec. 23, 2022, and U.S. Provisional Patent Application No. 63/511,132, filed Jun. 29, 2023, both of which are incorporated by reference herein in their entireties.

### TECHNICAL FIELD

The present technology generally relates to implantable medical devices and, in particular, to adjustable shunting systems for fluidly connecting a first body region and a second body region.

### BACKGROUND

Implantable shunting systems are widely used to treat a variety of patient conditions by shunting fluid from a first body region/cavity to a second body region/cavity. The flow of fluid through the shunting systems is primarily controlled by the pressure gradient across the shunt lumen and the geometry (e.g., size) of the shunt lumen. One challenge with conventional shunting systems is selecting the appropriate geometry of the shunt lumen for a particular patient. A lumen that is too small may not provide enough therapy to the patient, while a lumen that is too large may create new issues in the patient. Despite this, most conventional shunts cannot be adjusted once they have been implanted. Accordingly, once the system is implanted, the therapy provided by the shunting system cannot be adjusted or titrated to meet the patient's individual needs.

### BRIEF DESCRIPTION OF THE DRAWINGS

FIGS. 1A and 1B illustrate an adjustable shunting system in a deployed configuration and configured in accordance with select embodiments of the present technology.

FIG. 2 illustrates another adjustable shunting system in a deployed configuration and configured in accordance with select embodiments of the present technology.

FIG. 3 illustrates yet another adjustable shunting system in a deployed configuration and configured in accordance with select embodiments of the present technology.

FIG. 4 illustrates yet another adjustable shunting system in a deployed configuration and configured in accordance with select embodiments of the present technology.

FIG. 5 illustrates an actuator for use with an adjustable shunting system and configured in accordance with select embodiments of the present technology.

FIG. 6 illustrates another actuator for use with an adjustable shunting system and configured in accordance with select embodiments of the present technology.

FIGS. 7A and 7B illustrate aspects of an actuation assembly for use with an adjustable shunting system and configured in accordance with select embodiments of the present technology.

FIG. 8 illustrates another adjustable shunting system configured in accordance with select embodiments of the present technology.

### DETAILED DESCRIPTION

The present technology is directed to adjustable shunting systems for shunting fluid between a first body region and a second body region. In many of the embodiments described herein, the adjustable shunting systems include actuation assemblies that can be selectively manipulated after the system has been implanted in a patient to change a level of therapy provided by the system, such as to tailor the therapy to the patient's changing needs. The actuation assemblies can include a shape memory actuator having a plurality of leaflets or projections arranged relative to one another to form a generally annular structure with, e.g., a generally conical, frustoconical, funnel, and/or hyperboloid shape. The actuation assemblies can further include one or more membranes that individually jacket or cover individual projections of the plurality of projections to define a lumen extending through the shunt. As described in throughout this Detailed Description, individually jacketing the projections is expected to provide several advantages relative to shunts having lumens formed from a single membrane. As one skilled in the art will appreciate from the following Detailed Description, other aspects of the present technology may also provide additional advantages.

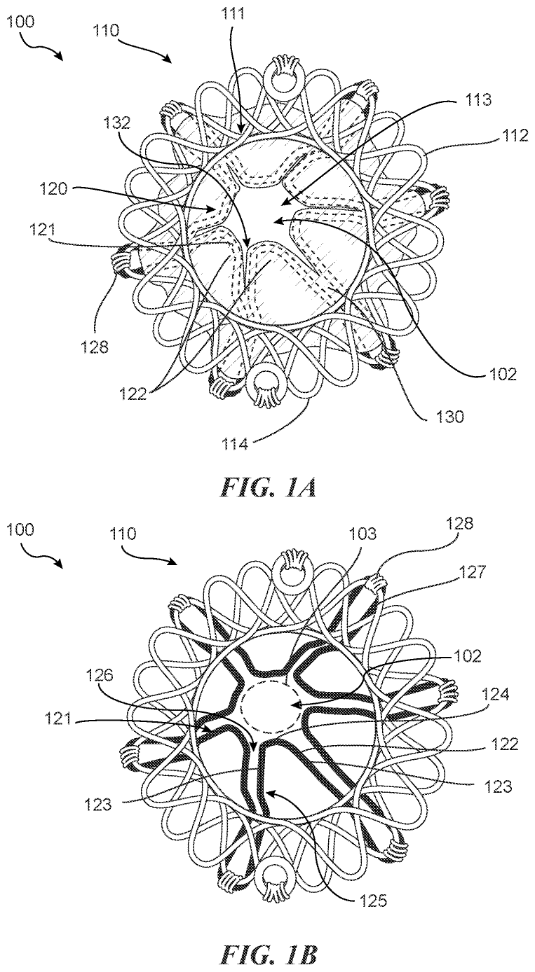

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to FIGS. 1A-8.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

As used herein, the use of relative terminology, such as “about”, “approximately”, “substantially” and the like refer to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art.

FIG. 1A illustrates an adjustable shunting system 100 (“the system 100”) in a deployed configuration and configured in accordance with select embodiments of the present technology. As described in detail below, the system 100 can be configured to shunt fluid between a first body region and a second body region when implanted in a patient (not shown). For example, the system 100 can be an interatrial shunting system configured to be implanted across a septal wall of a patient to shunt blood from the left atrium to the right atrium of the patient.

The system 100 includes an anchoring or stabilizing feature or structure 110 (“the anchor structure 110”) configured to secure the system 100 to patient tissue and/or stabilize the position of the system 100 in a desired anatomic location. In the illustrated embodiment, the anchor structure 110 is a wire or filament structure (e.g., a braided or woven wire structure) having a generally annular geometry. A radially inward portion 111 of the anchor structure 110 defines a central opening or passage 113. As described in detail below, an actuation assembly 120 can be coupled to the anchor structure 110 and sit at least partially within the opening 113 and/or extend from a perimeter of the radially inward portion 111. The actuation assembly 120 can define or at least partially define a lumen 102 extending through the opening 113, as described in greater detail below.

In the illustrated embodiment, the anchor structure 110 includes a first plurality of petals or appendages 112 and a second plurality of petals or appendages 114. In some embodiments, the wire forming pattern of the anchor structure 110 results in immediately adjacent petals of the first petals 112 not being formed by an adjacent segment of the wire structure forming the anchor structure 110. Instead, the wire structure can alternate between forming a first petal 112 on a first side of the system 100 and a second petal 114 on the second side of the system 100 (e.g., the portion of the wire structure that forms an individual first petal 112 at a 12:00 position may cross to the other side of the anchor structure 110 to form an individual second petal at a 3:00 position before crossing back to form another individual first petal 112 at a 5:00 position, and so on). The first plurality of petals 112 and the second plurality of petals 114 are separated by a gap (not shown).

When the system 100 is deployed across a tissue structure (e.g., a septal wall-not shown), the system 100 is configured to receive patient tissue between the first petals 112 and the second petals 114, e.g., in the gap. Additionally, the first plurality of petals 112 and the second plurality of petals 114 can be at least partially biased toward one another such that the first petals 112 and the second petals 114 at least partially squeeze patient tissue received within the gap to secure the system 100 to patient tissue. For example, when deployed across the septal wall, the first petals 112 may reside within the left atrium, the second petals 114 may reside within the right atrium, and the gap between the first petals 112 and the second petals 114 may receive a portion of the patient's septal wall (e.g., at the fossa ovalis). The first petals 112 may be biased at least slightly toward the second petals 114 (and/or the second petals 114 may be biased at least slightly toward the first petals 112) such that the anchor structure 110 forms a slight clamping force on the portion of the septal wall within the gap 118. In some embodiments, the first petals 112 and the second petals 114 are at least partially staggered such that individual first petals 112 do not entirely overlap with individual second petals 114. Without being bound by theory, this is expected to spread the pinching force over a larger area of the septal wall.

The anchor structure 110 can be at least partially composed of a self-expanding material such that, after being exposed to stress and strain induced by being collapsed into a delivery tool (e.g., catheter, sheath, etc.) for delivery, it exhibits an elastic response when being deployed at body temperature. For example, the anchor structure 110 can be composed, at least in part, of Nitinol that has an austenite finish temperature below body temperature. Accordingly, the anchor structure 110 can automatically deploy (e.g., self-expand without additional input or manipulation by a clinician) from a collapsed delivery configuration (e.g., as positioned in a delivery tool such as a catheter or sheath) to an expanded deployed configuration when released from the delivery tool. In some embodiments, the self-expanding or superelastic properties of the anchor structure 110 may also enable the anchor structure 110 to resist plastic mechanical deformation once deployed, and thus can provide a generally stable anchoring mechanism for the system 100. In other embodiments, the anchor structure 110 can be composed of a material that is not self-expanding at body temperature. In one example, the anchor structure 110 can be composed of Nitinol that has an austenite finish temperature above body temperature. In such an example, the anchor structure 110 can be initially released from a delivery tool in a preliminary position (e.g., the collapsed delivery configuration, an intermediate configuration, etc.) and subsequently be heated above the austenite finish temperature to transition the shape of the anchor structure 110 toward the deployed configuration. In a second example, the anchor structure 110 can be composed of a material such as stainless steel (e.g., 316L), titanium alloy (e.g., TiAl₆V₄), cobalt chromium alloy (e.g., L605), or polymer (e.g., PEEK). Some implementations of the second example can be self-expanding based upon a geometric configuration of the anchor structure 110. Other implementations can be manually expanded by an operator after an initial deployment using tools such as catheters, sutures, balloons, and the like. Regardless of its material composition, in some embodiments, some or all of the anchor structure 110 can include an insulative or coating material. Examples of suitable materials include, but are not limited to, perylene, urethane, ePTFE, or the like. In some embodiments, the anchor structure 110 can include multiple insulative/coating layers (e.g., perylene and urethane in alternating layers). The coating material can be selected to (a) improve the biocompatibility of the anchor structure 110, (b) improve the lubriciousness of the anchor structure 110, and/or (c) improve inductive properties of the anchor structure 110, as described in greater detail below. Additional details regarding anchoring features suitable for use with the system 100 are described in International Patent Application No. PCT/US2022/046584, the disclosure of which is incorporated by reference herein in its entirety.

The actuation assembly 120 includes an actuator 121 partially or fully covered by a membrane 130 that is fluidically impermeable or at least substantially fluidically impermeable to blood and/or other bodily fluids (the portion of the actuator 121 covered by the membrane 130 is shown in broken line in FIG. 1A). Together, the actuator 121 and the membrane 130 form a generally conical, frustoconical, funnel, cylindrical, or hyperboloid shape with an opening on both ends of the “cone.” In this way, the actuation assembly 120 at least partially defines the lumen 102 extending through the system 100, as described above. Accordingly, when the system 100 is implanted in a patient (e.g., across a septal wall of the patient), fluid can flow through the system 100 via the lumen 102 extending through the actuation assembly 120. As described in detail below, the actuation assembly 120 is configured to change one or more therapy parameters associated with the shunt (e.g., fluid resistance, lumen size, orifice size, flow rate, etc.) to control the therapy provided by the system 100. For example, the actuation assembly 120 can be transitioned between a plurality of unique positions or configurations, with each unique position or configuration providing a different fluid resistance through the lumen 102.

As best shown in FIG. 1B, which shows the system 100 with the membrane 130 omitted to more clearly illustrate aspects of the actuator 121, the actuator 121 can be formed via one or more wires or wire-like structures. As shown, for example, the actuator 121 can include a plurality of projections 122 (e.g., leaflets, fingers, wings, struts, petals, lobes, etc.) formed via the one or more wires or wire-like structures. The projections 122 can be formed to define the desired cylindrical, conical, frustoconical, funnel, and/or hyperboloid shape. In some embodiments, the plurality of projections 122 are formed from a single or common wire structure. In other embodiments, individual projections (or fewer than all of the plurality of projections) of the plurality of projections 122 can be formed by separate wire structures. Each projection 122 generally includes two struts 123 connected via a tip 124, thereby forming a general “U” or “V” shape. Each individual strut 123 is spaced apart from an individual strut 123 of the neighboring projection 122 by a gap 126. This at least partially mechanically separates neighboring projections 122 and enables individual projections 122 to be individually jacketed by discontinuous portions of the membrane 130, as described in detail below. Collectively, the tips 124 form a generally circular central aperture or opening 103 (the opening 103 is approximated in broken line in FIG. 1B) to the lumen 102. In variation embodiments, the tips 124 can form alternately shaped openings 103, for example generally square shapes, generally pentagonal or hexagonal shapes, oval shapes, etc. When the system 100 is implanted in a patient, fluid can flow through the opening 103 as it enters or exits the lumen 102. Although the illustrated embodiment shows the actuator 121 as having six projections 122, in other embodiments the actuator 121 can have fewer or more projections, such as two, three, four, five, seven, eight, nine, or more.

In some embodiments, the struts 123 can include a slight curvature or bend region 125. As described in detail below, the projections 122 can be configured to hinge or otherwise bend at the bend regions 125 when the actuation assembly 120 is transitioned to a different configuration to change a flow characteristic through the shunt. For example, to increase a dimension of the lumen 102 and/or the opening 103, and thus to decrease a fluid resistance through the system 100, the projections can be deflected radially outward by decreasing a degree of curvature at the bend region 125. Conversely, to decrease a dimension of the lumen 102 and/or the opening 103, and thus to increase a fluid resistance through the system 100, the projections can be deflected radially inward by increasing a degree of curvature at the bend region 125.

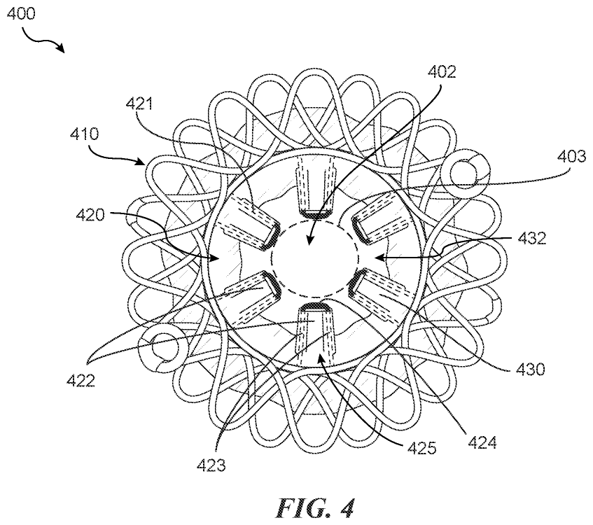

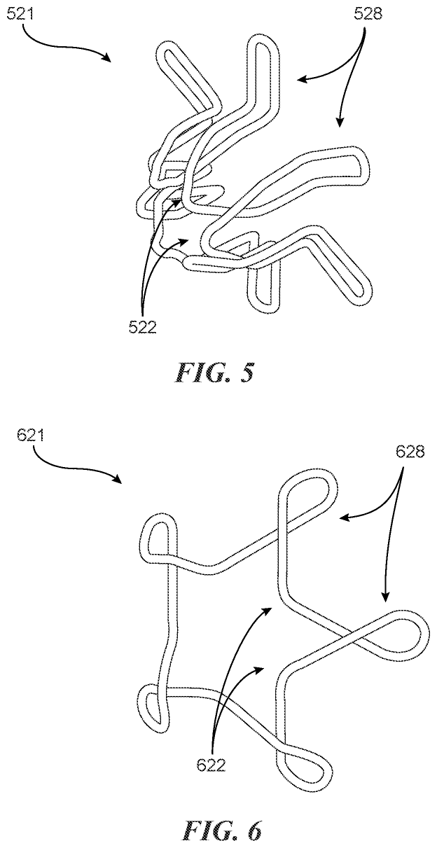

In some embodiments, the actuator 121 further includes a flange or waist region with a plurality of secondary projections 127 that can extend around or from a “base” of the projections 122 (additional details of actuators having flanges or secondary projections are described below with reference to FIGS. 5 and 6). The actuator flange/secondary projections 127 can be positioned adjacent to and in a common plane with the first petals 112 and/or the second petals 114 of the anchor structure 110, and thus can be coupled to the anchor structure 110 to secure the actuation assembly 120 thereto. For example, the actuation assembly 120 can be mechanically coupled to the anchor structure 110 at a plurality of connection points 128 (e.g., using sutures, crimps, glue, tape, micro-molded fasteners, etc.) positioned proximate tips of the first petals 112 and/or tips of the second petals 114. In other embodiments, the actuation assembly 120 can be coupled to the anchor structure 110 via other mechanisms and/or at other positions (e.g., proximate the radially inward portion 111 of the anchor structure 110, similar to the embodiment described below with reference to FIG. 4). Mechanically coupling the actuation assembly 120 to the anchor structure 110 is expected to stabilize the actuation assembly 120 within the opening 113.

To facilitate adjustment of the actuation assembly 120, the actuator 121 can be composed at least in part of shape memory material, such as Nitinol. The actuator 121 can therefore be transitionable at least between a first material phase or state (e.g., a martensitic state, a R-phase, a composite state between martensitic and R-phase, etc.) and a second material phase or state (e.g., an austenitic state, an R-phase state, a composite state between austenitic and R-phase, etc.). In the first material state, the actuator 121 may have reduced (e.g., relatively less stiff) mechanical properties that cause the actuator 121 to be more easily deformable (e.g., plastically compressible, expandable, etc.) relative to when the actuator is in the second material state. In the second material state, the actuator 121 may have increased (e.g., relatively more stiff) mechanical properties relative to the first material state, causing an increased preference toward a specific preferred geometry (e.g., original geometry, manufactured geometry, fabricated geometry, heat-set geometry, etc.). If the actuator 121 is deformed relative to its preferred geometry when in the first material state, heating the actuator 121 above its transition temperature causes the actuator 121 to move to and/or toward its preferred geometry as it transitions to its second material state. In some embodiments, the actuator 121 is fabricated to have a transition temperature greater than average body temperature such that, during and after implantation in a patient, the actuator 121 remains in the first material state. For example, the actuator 121 can have a transition temperature between about 38 degrees Celsius and about 80 degrees Celsius, or between about 40 degrees Celsius and about 65 degrees Celsius, or between about 40 degrees Celsius and about 55 degrees Celsius, or between about 45 degrees Celsius and about 50 degrees Celsius.

Referring again to FIG. 1A, in some embodiments the actuator 121 is configured such that its preferred/shape memory geometry, which can also be referred to herein as a “reset configuration,” defines a minimum treatment dimension for the lumen 102 (as used herein, the term “minimum treatment dimension” refers to a minimum dimension of the lumen 102 when the actuator 121 and the lumen 102 occupy a configuration intended for treatment—in some embodiments, the minimum treatment dimension may therefore be greater than a dimension of the lumen 102 when the actuator 121 is collapsed in a catheter and/or greater than a dimension of the lumen 102 shortly after the actuator 121 has been deployed from the catheter but before it has been expanded/reset into its preferred geometry). Accordingly, when the actuator 121 is in its preferred geometry, the lumen 102 provides the highest treatment-intended fluid resistance, such as by the opening 103 having a minimum deployed diameter. Because the actuator 121 has a transition temperature greater than body temperature and is therefore generally in the first material state at body temperature, the actuator 121 can be mechanically deformed at body temperature to change a dimension of the lumen 102 and/or opening 103. This can be done, for example, by positioning a catheter carrying an expandable element (e.g., a non-compliant balloon) within the lumen 102 and expanding the expandable member until the actuator 121 has been deformed (e.g., dilated or expanded) to a desired position. More specifically, expanding the expandible element may mechanically deflect the projections 122 radially outwardly (e.g., by decreasing a curvature at the bend regions 125) to increase a diameter of the opening 103, thereby decreasing resistance through the lumen 102. The expandable element can then be collapsed and retracted, but the actuator 121 can be configured to retain its deformed (e.g., dilated or expanded) state by virtue of the material properties associated with the first material state.

To decrease a dimension of the lumen 102 and/or opening 103, the actuator 121 can be heated above its transition temperature to transition to the second material state. Transitioning to the second material state causes the actuator 121 to “reset” to its preferred geometry (e.g., the “reset configuration”), which as set forth above can be associated with the minimum deployed dimension for the lumen 102. For example, thermally resetting the actuator 121 may cause the projections 122 to move radially inwardly (e.g., by bending or increasing a curvature at the bend regions 125) to decrease a diameter of the opening 103. In some embodiments, the actuator 121 can be thermally reset via resistive heating. For example, some or all of the anchor structure 110 can form an inductor for generating electrical energy in response to being exposed to an electromagnetic field. The anchor structure 110 can be electrically coupled to the actuator 121 such that the electrical energy generated in the anchor structure 110 flows into the actuator 121 and resistively heats the actuator 121, e.g., above its transition temperature. Additional details regarding incorporating shape memory actuators into RLC circuits and using anchor structures as inductors are described below with reference to FIGS. 3A and 3B, and in International Patent Application Publication Nos. WO 2022/076601 and WO 2022/081980, the disclosures of which are incorporated by reference herein in their entireties.

Once reset to its preferred geometry and cooled below the transition temperature (e.g., by returning to body temperature), a user can optionally once again mechanically expand the actuator 121 to a desired dimension. Thus, the actuation assembly 120 can be repeatedly and selectively manipulated by a user to adjust the therapy level provided by the system 100. Additional examples of and details for operating shape memory actuators for adjustable shunts are described in U.S. Patent Application Publication Nos. 2021/0085935 and 2022/0142652, the disclosures of which are incorporated by reference herein in their entireties.

Referring again to FIG. 1A and as set forth above, the actuation assembly 120 includes a membrane 130 covering (e.g., jacketing) the projections 122. The membrane 130 creates/defines walls of the lumen 102, and therefore defines the flow path by which fluid may travel through the system 100. Without such a membrane defining or at least partially defining a flow channel through the shunting device, dimensional changes of the lumen 102 and/or opening 103 described above would have limited impact on the characteristics of flow through the system 100. Of note, individual projections 122 are covered by individual membranes 130 or individual membrane portions such that a mechanical separation 132 exists between adjacent projections 122. In embodiments in which individual projections 122 are covered by individual membrane portions as opposed to individual membranes, the membrane 130 may be composed of a single piece of material but can have cut outs or slits corresponding to the separation 132 between the individual projections 122. As used herein, the term “individually jacketing” therefore includes both covering individual projections 122 with separate individual membranes 130 and covering individual projections 122 with individual portions of a common membrane.

Individually jacketing individual projections 122 is expected to be advantageous because it restricts motion of the projections 122 to a lesser degree as compared to if the projections 122 were covered by a single, contiguous membrane (e.g., if there were no mechanical separations or discontinuities between membrane portions covering adjacent projections 122). This is because the membrane 130 and the projections 122 do not need to move relative to one another as the actuation assembly 120 transitions between various configurations. As a result, the membrane 130 does not need to be composed of a flexible or “stretchy” material and/or be comprised of an oversized (relative to the actuation assembly 120) material to accommodate movement of the projections 122 during actuation of the actuation assembly 120, as described above. Rather, the membrane 130 can move with the individual projections 122 as they are deflected radially outwardly or reset radially inwardly. Individually jacketing the projections 122 also enables individual projections 122 to slide/move relative to each other without needing to substantially stretch the membrane 130. Further, in many embodiments individually jacketing the projections 122 removes or minimizes a constant radial inward force and/or hoop stress that would be applied to the projections 122 by a single, contiguous membrane when the geometry of the lumen 102 and/or opening 103 has been enlarged beyond the reset configuration. Still further, in many embodiments individually jacketing the projections 122 removes or minimizes the folding or buckling regions that would exist in a single contiguous membrane covering projections 122 when the geometry of the lumen 102 and/or opening 103 is in a relatively small or narrow configuration compared to the maximumly large or wide configuration that is possible.

For at least the foregoing reasons, individually jacketing the projections 122 is expected to increase the types of materials that can be used for the membrane 130. For example, the membrane 130 can be composed of materials that are generally stiffer but have favorable biocompatible properties, such as ePTFE. In other embodiments, the membrane 130 can be composed at least in part of other suitable materials, such as PTFE, PET, silicone, urethane, nylon, or the like, or a combination of suitable materials. In some embodiments, the membrane 130 is composed of ePTFE with a urethane coating. Because individually jacketing the projections 122 also reduces the amount the membrane 130 is stretched, individually jacketing the projections 122 is also expected provide advantages even if a generally stretchy or elastic material is used for the membrane 130. For example, individually jacketing the projections 122 is expected to reduce unwanted wrinkling, tenting, tearing, and/or other deformations that could occur by repeatedly stretching the membrane 130.

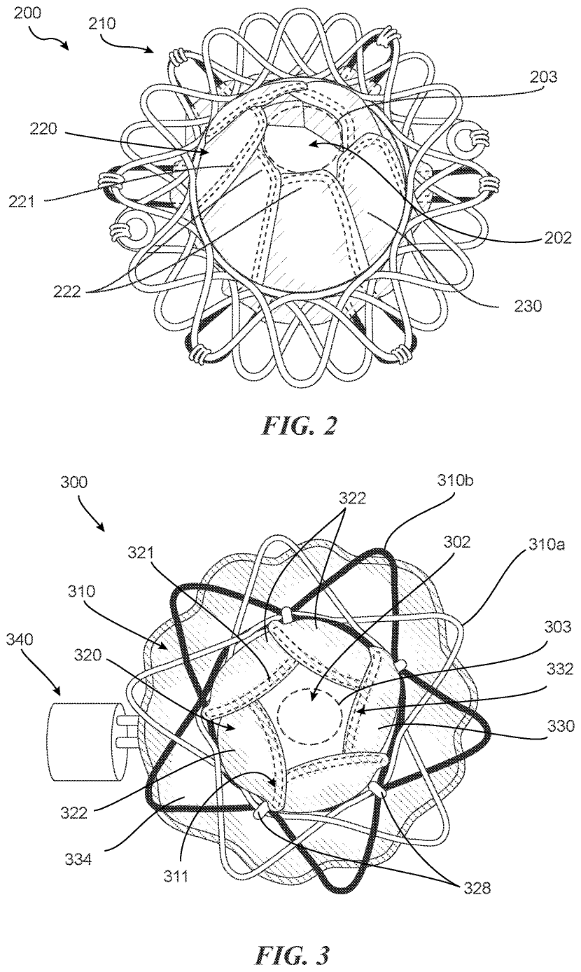

FIG. 2 illustrates another adjustable shunting system 200 (“the system 200”) in a deployed configuration and configured in accordance with select embodiments of the present technology. The system 200 can include certain features generally similar to the features of the system 100 of FIGS. 1A and 1B. For example, the system 200 can include an anchor structure 210, which can be generally similar to or the same as the anchor structure 110 of the system 100. The system 200 can also include an actuation assembly 220 having an actuator 221 and a membrane 230 and defining a lumen 202 with an opening 203. Similar to the system 100 described with reference to FIGS. 1A and 1B, the actuator 221 can include a plurality of projections 222 composed of a shape memory material for facilitating adjustment of the actuation assembly 220. The projections 222 are individually jacketed by the membrane 230 or portions of the membrane 230 to reduce the resistance the membrane 230 provides when the actuator 121 is adjusted.

Relative to the system 100 of FIGS. 1A and 1B, the projections 222 of the actuation assembly 120 at least partially overlap when the actuator 121 is in its preferred geometry/reset configuration (e.g., after the actuator 121 has been reset to its base configuration by heating the actuator 121 above its transition temperature). That is, individual projections 222 at least partially overlap neighboring projections 222. In some embodiments, when the actuator 221 is in its preferred geometry/reset configuration, neighboring projections 222 may overlap by between about 0.1 mm and 2.5 mm, or between about 0.1 mm and about 2 mm, or between about 0.1 mm and about 1.5 mm, or between about 0.5 mm and about 1.5 mm, or between about 0.5 mm and about 1.0 mm, or between about 0.1 mm and about 1.0 mm, or between about 0.1 mm and about 0.5 mm. For example, in some embodiments neighboring projections 222 overlap by about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, or about 2.5 mm. The foregoing ranges and values are provided by way of example only-in other embodiments, the projections 222 may overlap more or less. Moreover, it is expected that the amount of overlap will vary along the length of the projections 222. For example, in the illustrated embodiment there is more overlap proximate a base of the projections 222 than proximate a tip of the projections 222. In some embodiments, this could be reversed, such that there is more overlap proximate the tip of the projections 222 than proximate the base of the projections 222. Regardless, unless the context clearly indicates otherwise, the use of specific dimensions in the context of overlapping petals refers to the maximum amount of overlap between two neighboring projections 222 at any point along their length.

In some embodiments, the projections 222 are configured to overlap when the actuator 212 is in the reset (e.g., narrowest) configuration but not when the actuator 221 is in a dilated/expanded (e.g., wider) configuration. In other embodiments, the projections 222 can be configured to overlap when the actuator is in the reset configuration and in some, but not all, of the potential dilated configurations. In yet other embodiments, the projections 222 can be configured to overlap when the actuator is in the reset configuration and in all of the potential dilated configurations. Regardless, one skilled in the art will appreciate that the amount of overlap between neighboring projections 222 will vary based on the configuration of the actuator 221.

Without being bound by theory, overlapping projections 222 may provide at least four advantages relative to non-overlapping projections. First, overlapping projections 222 may reduce the amount of fluid that can flow (e.g., leak) between neighboring projections 222. Instead, fluid is more likely to flow through the opening 203 and the lumen 202. Second, overlapping projections 222 may at least partially mechanically couple a plurality of projections 222 and thereby provide stability to the overall shape of the opening 203 during movement of the actuator 221, especially during mechanical expansion of the actuator 221. For example, during a balloon expansion of the actuation assembly 220 to decrease fluid resistance through the lumen 202, an individual projection 222 that is being pushed radially outwardly by the balloon will apply a mechanical, radially outward force against at least one neighboring projection 222 that it overlaps with. As a result, it is expected that all of the projections 222 will expand radially outward in uniform, even if the balloon does not directly contact each projection 222. Third, overlapping projections 222 may be simpler to collapse into a delivery configuration that can fit within a catheter. In other words, the overlapping projections 222 may simplify the process of folding or otherwise collapsing the system 200 such that it can fit within a catheter that can be percutaneously advanced to a target location within the patient for deployment. Fourth, overlapping projections 222 may make it easier to advance a percutaneous tool (e.g., a balloon, catheter, guidewire, etc.) through the corresponding opening 203 and lumen 202 because it will reduce the possibility of the tool getting caught between adjacent projections. Of course, other advantages of overlapping projections may exist beyond those expressly identified herein, and the present technology is not limited by the foregoing advantages.

FIG. 3 illustrates another adjustable shunting system 300 (“the system 300”) in a deployed configuration and configured in accordance with select embodiments of the present technology. The system 300 can include certain features generally similar to the features of the system 100 of FIGS. 1A and 1B and the system 200 of FIG. 2. For example, the system 300 can include an anchor structure 310 configured to stabilize the system 300 across a target anatomical structure (e.g., a septal wall between a left atrium and a right atrium). However, relative to the systems 100 and 200 of FIGS. 1A-2, the anchor structure 310 includes a first wire portion 310a and a second wire portion 310b. The first wire portion 310a and the second wire portion 310b can be composed at least in part of a common material, such as Nitinol set to have superelastic properties at body temperature. However, the first wire portion 310a can further include a conductive cladding/coating (e.g., silver or copper cladding) surrounding the Nitinol core, whereas the second wire portion 310b does not include the conductive cladding. In alternate embodiments, the conductive portion of the material can be interior to a Nitinol shell. In further variations, the conductive material may be combined with or interfaced with materials other than Nitinol to comprise a hybrid material structure. In such embodiments, the first wire portion 310a can therefore have more favorable electrical properties to function as an inductor or antenna to wirelessly receive energy transmissions (e.g., to resistively heat the actuator 321 and/or to charge one or more active components of the system 300 such as sensors, other electronics, etc.), while the second wire portion 310b can function with more favorable mechanical properties (e.g., stronger superelasticity) to mechanically stabilize the system 300. In some embodiments, the first wire portion 310a and/or second wire portion 310b can interface with other components (e.g., capacitors, inductors, resistors, microcontrollers, etc.) to aid in the functions of acting as an inductor or antenna. In some embodiments, the first wire portion 310a and the second wire portion 310b are part of the same wire. In other embodiments, the first wire portion 310a and the second wire portion 310b are part of different wires (e.g., the first wire portion 310a and the second wire portion 310b are discrete wires, e.g., a first wire 310a and a second wire 310b). In some embodiments, the first wire portion 310a and the second wire portion 310b are not electrically connected (e.g., not electrically in series) such that current generated in the first wire portion 310a does pass through the second wire portion 310b. Indeed, in some embodiments the first wire portion 310a may form part of an electrical circuit with one or more system components (e.g., the actuator 321), and the second wire portion 310b may form an open circuit terminating at an electrically insulated end of the second wire portion 310b (e.g., in an epoxy filled potted cap structure, not shown). Additional details regarding using stabilizing/anchoring features as inductors are described in International Patent Application Publication No. WO 2022/081980, previously incorporated by reference herein.

The actuation assembly 320 can also include certain features generally similar to those described with reference to the actuation assemblies 120, 220 described with reference to FIGS. 1A-2. For example, the actuation assembly 320 can include the actuator 321 and a membrane 330 that together define a lumen 302 with an opening 303. The actuator 321 can include a plurality of petals or projections 322 composed of a shape memory material for facilitating adjustment of the actuation assembly 320. Similar to the system 200 described with reference to FIG. 2, the neighboring projections 322 at least partially overlap. Also similar to the systems 100 and 200 described with reference to FIGS. 1A-2, the projections 322 are individually jacketed by the membrane 330. As shown, the membrane 330 includes a plurality of first membrane portions 332 (e.g., a shirt) that jacket corresponding individual projections 322. The membrane 330 also includes a “skirt” comprising one or more second membrane portions 334 that jacket or otherwise at least partially cover a portion of the actuator 321 that abuts the anchor structure 310 (see, e.g., FIGS. 5 and 6). Moreover, unlike the system 100 and the system 200, the actuation assembly 320 is coupled to a radially inward portion or “waist” 311 of the anchor structure 310. For example, the actuation assembly 320 is coupled to the radially inward portion 311 via a plurality of connection elements 328 (e.g., sutures, ties, tape, glue, crimps, molded fasteners, etc.).

The actuator 321 can be electrically coupled to the first wire portion 310a of the anchor structure 310. For example, the system 300 can include an electrical connector subassembly 340 at which an end portion of the first wire portion 310a is electrically coupled to an end portion of the actuator 321. In some embodiments, the electrical connector subassembly 340 includes a capacitor (not shown) electrically coupled between the first wire portion 310a and the actuator 321. For example, the end portion of the first wire portion 310a can be crimped, soldered, or otherwise coupled to a first terminal of the capacitor, and the end portion of the actuator 321 can be crimped, soldered, or otherwise coupled to a second terminal of the capacitor. The capacitor and the corresponding connections between the capacitor and the first wire portion 310a and the actuator 321 can be embedded within an epoxy or other suitable material and positioned within a cap (e.g., a titanium cap), thereby forming a potted connection between the first wire portion 310a and the actuator 321. In some embodiments, the entire electrical connector subassembly 340 can be hermetically sealed, e.g., in addition to or instead of embedding the capacitor into an epoxy.

In some embodiments, the actuator 321 and the first wire portion 310a are electrically disconnected from the second wire portion 310b of the anchor structure 310. That is, the electrical circuit formed by the first wire portion 310a and the actuator 321 does not include the first wire portion 310b. Instead, as set forth above the second wire portion 310b may form an “open” circuit that terminates at an insulated end of the second wire portion 310b, such as in a second epoxy filled potted cap structure (not shown), different than the electrical connector subassembly 340. Excluding the second wire portion 310b from the electrical circuit that includes the actuator 321 may be advantageous because it may reduce the total resistance of the circuit in the anchor structure 310 and provide more efficient energy transfer to the actuator 321.

To actuate the actuator 321, a user can generate an electromagnetic field surrounding and/or directed at the first wire portion 310a (e.g., via a transmit coil positioned at a distal end portion of a catheter extending proximate to the first wire portion 310a). Due to its inductive properties, the first wire portion 310a generates electrical energy in response to being exposed to the electromagnetic field. The generated electrical energy can flow through the first wire portion 310a, through the capacitor (not shown) at the electrical connector subassembly 340, and into the actuator 321. The electrical energy flowing through the actuator 321 can resistively heat the actuator 321. Accordingly, the first wire portion 310a, the capacitor of the electrical connector subassembly 340, and the actuator 321 form an RLC circuit, with the first wire portion 310a acting as the inductor and the actuator 321 acting as the resistor. In some embodiments, the RLC circuit is configured to ensure that electrical energy flows in the same direction (e.g., clockwise through the anchor structure 310 and the actuator 321). Additional details regarding incorporating shape memory actuators into RLC circuits are described in International Patent Application Publication No. WO 2022/076601, previously incorporated by reference herein.

Similar to the process described above for adjusting the actuator 121 of FIGS. 1A and 1B, heating the actuator 321 above its transition temperature can “reset” the actuator 321 to its preferred geometry, which can be associated with its minimum deployed dimension for the lumen 302. Once reset to its preferred geometry and cooled below the transition temperature, a user can optionally mechanically expand the actuator 321 to a desired dimension, e.g., to achieve a desired fluid resistance and/or flow rate through the lumen 302.

FIG. 4 illustrates yet another adjustable shunting system 400 (“the system 400”) in a deployed configuration and configured in accordance with select embodiments of the present technology. The system 400 can include certain features generally similar to the features of the systems 100-300 of FIGS. 1A-3. For example, the system 400 can include an anchor structure 410 that can be generally similar to or the same as the anchor structure 110 of the system 100. The system 400 can also include an actuation assembly 420 having an actuator 421 and a membrane 430 defining a lumen 402 with an opening 403. Similar to the systems 100-300 described with reference to FIGS. 1A-3, the actuator 421 can include a plurality of projections 422 composed of a shape memory material for facilitating adjustment of the actuation assembly 420.

However, relative the system 200 of FIG. 2 and the system 300 of FIGS. 3A and 3B, neighboring projections 422 are spaced apart by a gap 432. And compared to the system 100 of FIGS. 1A and 1B, the gap 432 between neighboring projections 422 is substantially larger. For example, a widest portion of the gap 432 can be at least 0.5 mm, at least 1 mm, at least 1.5 mm, or at least 2 mm. Moreover, relative to the system 100 of FIGS. 1A and 1B, the projections 422 of the system 400 can be formed by substantially parallel struts 423. As a result, the width of the gap 432 decreases toward a tip 424 of the projections 422 as compared to the width of the gap at a base 425 of the projections 422, while a width of the projections 422 remains generally constant across its length. One possible advantage of the actuator 421 is that it may allow relatively more flow through the system 400 compared to the actuators 321, 221, 121 described with reference to FIGS. 1A-3 when the struts are in comparable angular configurations. A second possible advantage of the actuator 421 is that the ability to deform the geometry of struts 423 may be less impacted by tissue overgrowth relative to other previously discussed configurations (e.g., any tissue overgrowth is more likely to be confined to individual projections 422, rather than growing between/across neighboring projections 422, which may inhibit movement of the projections 422).

Although each of the actuators 121-421 are shown as having generally symmetrical projections/petals, in variation embodiments, the actuator struts forming the projections/petals, and/or the projections/petals themselves, may not be symmetrically configured. For example, some embodiments may utilize one or more relatively larger struts or projections and one or more relatively smaller struts or projections (e.g., at least one pair of asymmetrical struts). Some embodiments may utilize alternatingly-sized struts or projections, and/or may utilize struts with different mechanical properties (e.g., deriving from different wire thickness) in different sections of the lumen openings. Such embodiments may make the actuators more resistant to unintentional deformations, for example deformations induced via incidental contact of an adjustment tool (e.g., a balloon catheter) during removal of the tool from the body. In other variations, other aspects of the actuator may vary and/or be asymmetric—for example the spacings between struts, the strut jacket material and/or thickness or density, the strut height, etc.

Moreover, although each of the actuators 121-421 of FIGS. 1A-4 is shown as a separate component from the corresponding anchor structures 110-410, in some embodiments the actuators 121-141 can be part of the same component as the anchor structure such that no specific coupling mechanism (e.g., connection points 128 in FIGS. 1A and 1B) is necessary. For example, in some embodiments any of the actuators 121-421 and the corresponding anchor structures 110-410 can be formed by a common Nitinol or other shape memory wire. In such embodiments, the portion of the common wire forming the actuator 121-141 can be heat-treated to exhibit shape memory properties at body temperature (e.g., set to have a phase transformation temperature greater than body temperature) and the portion of the common wire forming the anchor structure 110 can be heat-treated to exhibit superelastic properties at body temperature (e.g., set to have a phase transformation temperature less than body temperature).

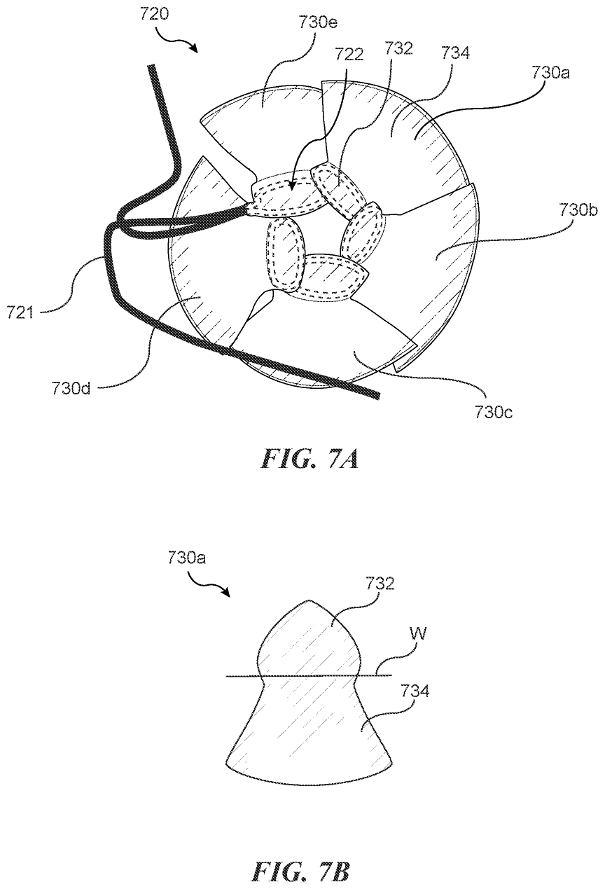

The systems described herein can have actuators other than those shown in FIGS. 1A-4 that enable petals/projections on the actuators to be individually jacketed by a corresponding membrane or membrane portion. For example, FIG. 5 illustrates an actuator 521 configured in accordance with select embodiments of the present technology. The actuator 521 can function generally similarly to the actuators 121, 221, 321, and 421 described with reference to FIGS. 1A-4, and therefore can be used in place of the foregoing actuators with any of the systems 100-400. For example, similar to the actuators described above, the actuator 521 is formed of one or more wires or wire-like structures, and includes a plurality of projections 522 that, when the actuator 521 is coupled to an anchor structure (e.g., the anchor structure 110 of the system 100), define a lumen. The actuator 521 further includes a plurality of secondary projections 528. The secondary projections 528 occupy a different plane than the projections 522 and can be used to couple the actuator 521 to the corresponding anchor structure (not shown in FIG. 5), such as at connection points 128 (FIG. 1A) if the actuator 521 is used with the system 100 of FIGS. 1A and 1B. Accordingly, the secondary projections 528 can be designed to abut a portion of the anchor structure (e.g., the tips of the first petals 112 and/or the second petals 114 of the anchor structure 110 of FIGS. 1A and 1B) and be mechanically coupled thereto (e.g., via sutures, glue, crimps, molded fasteners, etc.) to stabilize the actuator 521 to the anchor structure. In some embodiments, the secondary projections 528 are configured to reside on an opposite side of an anatomical structure from a tip of the projections 522 when the actuator 521 is implanted in a patient as part of an adjustable shunting system. For example, in embodiments in which the actuator 521 is used with an adjustable interatrial shunting system configured to be implanted across a septal wall of a patient, the secondary projections 528 can be positionable in the left atrium and the tip of the projections 522 can be positioned in the right atrium, or vice versa. In some embodiments, the secondary projections 528 can be at least partially covered by a portion of the membrane covering the corresponding projection 522, such as with the skirt 334 of the membrane 330 described with reference to FIG. 3 and/or as described in greater detail below with reference to FIGS. 7A and 7B.

FIG. 6 illustrates yet another actuator 621 configured in accordance with select embodiments of the present technology. The actuator 621 can function generally similarly to the actuators 121, 221, 321, and 421 described with reference to FIGS. 1A-4, and therefore can be used in place of the foregoing actuators with any of the systems 100-400. Similar to the actuators described above, the actuator 621 is formed of one or more wires or wire-like structures, and includes a plurality of petals or projections 622 that, when the actuator 621 is coupled to an anchor structure (e.g., the anchor structure 110 of the system 100), define a lumen. The actuator 621 further includes a plurality of loops 628. The loops 628 can be used to couple the actuator 621 to the corresponding anchor structure (not shown in FIG. 6), such as at connection elements 328 (FIG. 3) if the actuator 621 is used with the system 300 of FIGS. 3A and 3B. Accordingly, the loops 628 can abut a portion of the anchor structure (e.g., the radially inward portion 111 of the anchor structure 110 of FIGS. 1A and 1B) and be mechanically coupled thereto (e.g., via sutures, glue, crimps, molded fasteners, etc.) to stabilize the actuator 621 to the anchor structure. When the actuator 621 is deformed relative to its preferred/shape memory geometry (e.g., during balloon expansion to increase a dimension of the actuator 121 beyond its reset configuration), strain builds in the loops 628 (e.g., as opposed to strain building in a designed bend region in the actuator, such as with the actuator 121 of FIGS. 1A and 1B). In some embodiments, the loops 628 can be covered or jacketed by a portion of the membrane covering the corresponding projection 622, such as with the skirt 334 of the membrane 330 described with reference to FIG. 3 and/or as described in greater detail below with reference to FIGS. 7A and 7B.

The systems described herein can have membranes other than those shown in FIGS. 1A-4. For example, FIG. 7A illustrates an actuation assembly 720 that can be used with any of the systems described herein. Similar to the actuation assemblies described herein, the actuation assembly 720 includes an actuator 721 having a plurality of projections 722, and a membrane 730 individually jacketing the plurality of projections 722. FIG. 7A illustrates the actuation assembly 720 during a manufacturing stage after the projections 722 have been formed and covered by the membrane 730, but before the remainder of the wire structure forming the actuator 721 has been set to a desired shape for coupling to a corresponding anchor structure (e.g., before secondary projections, such as the secondary projections 528 described with reference to the actuator 521 of FIG. 5 or the loops 628 described with reference to the actuator 621 of FIG. 6, have been formed in the actuator 721). As illustrated, the membrane 730 includes a plurality of individual membranes 730a-730e. Each individual membrane 730 includes a first membrane portion 732 (e.g., a shirt) and a second membrane portion 734 (e.g., a skirt). FIG. 7B illustrates a flat pattern of a single membrane 730a and illustrates the first membrane portion 732 and the second membrane portion 734 divided at a waist W.

Referring collectively to FIGS. 7A and 7B, the first membrane portion 732 of each membrane 730 jackets a corresponding individual projection 722. Accordingly, the first membrane portion 732 can be composed of two layers forming a pouch for receiving the projection 722. The second membrane portion 734 is configured to at least partially cover a portion of the actuator 721 configured to abut the corresponding anchor structure once the actuator 721 is fully formed (e.g., secondary projections for coupling to the anchor structure, such as the secondary projections 528 described with reference to the actuator 521 of FIG. 5 or the loops 628 described with reference to the actuator 621 of FIG. 6). Of note, in the illustrated embodiment the second membrane portion 734 is composed of a single layer and therefore does not fully jacket the portion of the actuator 721 configured to about the corresponding anchor structure. Without intending to be bound by theory, membranes that have a first portion that is double layered (e.g., the first membrane portion 732) and a second portion that is single layered (e.g., the second membrane portion 734) may be easier to install over the projections 722 because they define a pouch with a slot or opening that the projections 722 can be inserted through. As described above, however, in some embodiments the membranes described herein can fully jacket the projections and the portion of the actuator configured to abut the anchor structure.

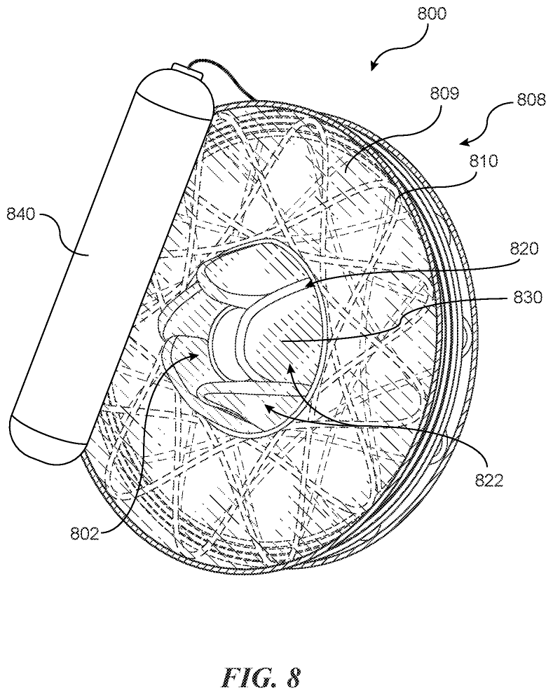

FIG. 8 illustrates another embodiment of an adjustable shunting system 800 (“the system 800”) configured in accordance with select embodiments of the present technology. The system 800 can include certain features generally similar to the features of the systems 100-400 of FIGS. 1A-4 described in detail above. For example, the system 800 can include an anchor assembly 808 for anchoring the system 800 at a desired anatomical location (e.g., across a septal wall of a patient). The system 800 can also include an actuation assembly 820 having a plurality of projections or petals 822 covered by one or more discontinuous membranes 830, as described in detail above with reference to FIGS. 1A-7. The actuation assembly 820 can form a lumen 802 extending therethrough, e.g., to shunt fluid between a first body region and a second body region when the system 100 is implanted in the patient.

Relative to the systems described with reference to FIGS. 1A-4, the anchor assembly 808 includes an anchor structure 810 covered by (e.g., disposed within) an anchor membrane 809 (“the membrane 809”). In some embodiments, the membrane 809 fully encases the anchor structure 810, e.g., such that the anchor structure 810 is not directly exposed to bodily fluids when the system 800 is implanted. For example, the membrane 809 can be a laminate structure comprising two sheets of material adhered together with the anchor structure 810 positioned therebetween, a single piece of material folded over to encase the anchor structure 810, or have other suitable configurations. The membrane 809 can be composed of a biocompatible and/or anti-thrombogenic material. Example materials include, but are not limited to, ePTFE, PTFE, PET, silicone, urethane, nylon, and the like.

In some embodiments, the membrane 809 that covers the anchor structure 810 is different than (e.g., not integral with) the one or more membranes 830 covering the projections 822 of the actuation assembly 820. Accordingly, the membrane 809 can optionally be composed of a different material than the membrane(s) 830. In other embodiments, the membrane 809 can be integral with one or more of the membrane(s) 830 covering the projections 822.

The system 800 can also optionally include a canister 840 coupled to the anchor structure 810 or another portion of the system 800. The canister 840 can be a sealed (e.g., hermetically sealed) container that houses various electronics and other components of the system 800. For example, the canister 840 can house one or more energy storage components (e.g., a primary cell battery, a rechargeable battery, a capacitor, a supercapacitor, etc.), one or more sensors or associated electronic circuitry (e.g., pressure sensor, flow sensor, etc.), one or more data storage elements (e.g., memory), one or more processors, one or more telemetry components, one or more microcontrollers, or the like. The canister 840 can be composed of a generally rigid material, such as titanium, steel, plastic, or the like. The canister 840 may also be covered by a biocompatible membrane composed of, for example, ePTFE or another suitable material. Although shown as having a single canister 840, in other embodiments the system 800 can have additional canisters, such as two, three, four, or more.

As one of skill in the art will appreciate from the disclosure herein, various components of the systems described above can be omitted without deviating from the scope of the present technology. Likewise, additional components not explicitly described above may be added to the systems without deviating from the scope of the present technology. Moreover, the features described herein can be incorporated into other types of implantable medical devices beyond shunting systems. Representative other implantable medical devices include, but are not limited to, occlusion devices (e.g., septal occluders), septal sensor devices with a transeptal access port, stents (e.g., perfusion stents), valves, or the like. Accordingly, the present technology is not limited to the configurations expressly identified herein, but rather encompasses variations and alterations of the described systems.

### Examples

Several aspects of the present technology are set forth in the following examples:
1. A shunting system for fluidly connecting a first body region and a second body region of a patient, the system comprising:
   an anchor structure configured to stabilize the shunting system across a target anatomical structure; and
   an actuation assembly coupled to the anchor structure, wherein the actuation assembly includes—
      an actuator composed of one or more shape memory wires, the actuator including a plurality of projections that together define a cylindrical or conical shape having a lumen extending therethrough, wherein tips of the projections define an opening to the lumen, and
      a membrane covering the plurality of projections, wherein the membrane individually covers individual projections of the plurality of projections,
   wherein the actuation assembly is selectively transitionable between at least two or more configurations, and wherein the at least two or more configurations are associated with different fluid resistances through the lumen.
2. The system of example 1 wherein the at least two or more configurations include a reset position and one or more expanded configurations.
3. The system of example 2 wherein:
   when the actuation assembly is in the reset position, the lumen has a first resistance, and
   when the actuation assembly is in one of the one or more expanded configurations, the lumen has a second resistance,
   wherein the first resistance is greater than the second resistance.
4. The system of example 2 or example 3 wherein:
   when the actuation assembly is in the reset position, the opening has a first diameter, and
   when the actuation assembly is in one of the one or more expanded configurations, the opening has a second diameter,
   wherein the first diameter is less than the second diameter.
5. The system of any of examples 2-4 wherein:
   the actuator is configured to transition between (a) a first material state having relatively less stiff mechanical properties and (b) a second material state having relatively stiffer mechanical properties in response to being heated above a transition temperature that is greater than body temperature, and
   the actuation assembly is configured to return to the reset position in response to being heated above the transition temperature.
6. The system of any of examples 2-5 wherein, when the actuation assembly is in the reset position, neighboring projections of the plurality of projections at least partially overlap.
7. The system of example 6 wherein neighboring projections overlap by between about 0.1 mm and about 2.5 mm.
8. The system of example 6 wherein neighboring projections overlap by between about 0.5 mm and about 1.5 mm.
9. The system of any of examples 2-8 wherein neighboring projections of the plurality of projections at least partially overlap in both the reset position and the one or more expanded positions.
10. The system of any of examples 1-6 wherein neighboring projections of the plurality of projections are separated by a gap.
11. The system of example 10 wherein the gap is at least about 0.5 mm.
12. The system of example 10 wherein the gap is at least about 1 mm.
13. The system of any of examples 1-12 wherein the membrane includes a plurality of individual membranes, and wherein individual membranes of the plurality of membranes jacket individual projections of the plurality of projections.
14. The system of example 13 wherein the individual membranes each include a first membrane portion and a second membrane portion divided at a waist, and wherein the first membrane portion jackets an individual projection and the second membrane portion extends over a portion of the anchor structure.
15. The system of example 14 wherein the first membrane portion includes two layers and the second membrane portion includes a single layer.
16. The system of example 14 wherein the first membrane portion includes two layers and the second membrane portion includes two layers.
17. The system of any of examples 1-12 wherein the membrane includes a single membrane having a plurality of membrane portions separated by gaps, and wherein individual membrane portions of the plurality of membrane portions cover individual projections of the plurality of projections.
18. The system of any of examples 1-17 wherein the membrane is composed of ePTFE.
19. The system of any of examples 1-18 wherein the adjustable shunting system is an interatrial shunting system, and wherein the target anatomical structure is a septal wall of a heart between a left atrium and a right atrium.
20. The system of any of examples 1-19 wherein the membrane is a first membrane, and wherein the system further comprises a second membrane covering the anchor structure.
21. The system of example 20 wherein the first membrane and the second membrane are discontinuous.
22. The system of example 20 or example 21 wherein the second membrane is composed of a biocompatible and/or anti-thrombogenic material.
23. The system of any of examples 1-22 wherein the anchor structure is electrically coupled to the actuator via a capacitor.
24. The system of example 23 wherein the anchor structure, the actuator, and the capacitor form an RLC circuit.
25. A shunting system for fluidly connecting a first body region and a second body region of a patient, the system comprising:
   a plurality of petals each extending between a first end portion and a second end portion, wherein the petals are arranged in a cylindrical or conical shape to form a lumen therethrough, with an opening to the lumen defined by the first end portions of the petals;
   wherein each individual petal of the plurality of petals includes a corresponding individual membrane or individual membrane portion such that at least the first end portions of neighboring petals can move relative to one another without stretching the individual membranes or individual membrane portions.
26. The system of example 25 wherein the petals are configured such that the petals can flex radially inwardly and/or radially outwardly without stretching the membrane or membrane portions.
27. The system of example 26 wherein the opening size is adjustable by flexing the petals radially inwardly and/or radially outwardly.
28. The system of any of examples 25-27 wherein the membrane or membrane portion is stiff.
29. The system of any of examples 25-28 wherein neighboring petals of the plurality of petals overlap.
30. The system of any of examples 25-28 wherein neighboring petals of the plurality of petals are spaced apart by a gap.
31. The system of any of examples 25-30 wherein the plurality of petals include a shape memory wire extending therethrough.
32. An adjustable shunting system for fluidly connecting a first body region and a second body region of a patient, the system comprising:
   a plurality of membranes arranged in a conical or cylindrical shape to form a lumen therethrough,
   wherein each membrane has a perimeter including a first edge extending at least partially along a length of the lumen, a second edge extending at least partially along the length of the lumen, and a tip portion connecting the first edge and the second edge, wherein a width between the first edge and the second edge is less than a circumference of the lumen.
33. The system of example 32 wherein the width between the first edge and the second edge varies along a length of each membrane.
34. The system of example 32 or example 33 wherein neighboring membranes of the plurality of membranes at least partially overlap.
35. The system of example 32 or example 33 wherein neighboring membranes of the plurality of membranes are separated by a gap.

### Conclusion

Embodiments of the present disclosure may include some or all of the following components: a battery, supercapacitor, or other suitable power source; a microcontroller, FPGA, ASIC, or other programmable component or system capable of storing and executing software and/or firmware that drives operation of an implant; memory such as RAM or ROM to store data and/or software/firmware associated with an implant and/or its operation; wireless communication hardware such as an antenna system configured to transmit via Bluetooth, WiFi, or other protocols known in the art; energy harvesting means, for example a coil or antenna which is capable of receiving and/or reading an externally-provided signal which may be used to power the device, charge a battery, initiate a reading from a sensor, or for other purposes. Embodiments may also include one or more sensors, such as pressure sensors, impedance sensors, accelerometers, force/strain sensors, temperature sensors, flow sensors, optical sensors, cameras, microphones or other acoustic sensors, ultrasonic sensors, ECG or other cardiac rhythm sensors, SpO2 and other sensors adapted to measure tissue and/or blood gas levels, blood volume sensors, and other sensors known to those who are skilled in the art. Embodiments may include portions that are radiopaque and/or ultrasonically reflective to facilitate image-guided implantation or image guided procedures using techniques such as fluoroscopy, ultrasonography, or other imaging methods. Embodiments of the system may include specialized delivery catheters/systems that are adapted to deliver an implant and/or carry out a procedure. Systems may include components such as guidewires, sheaths, dilators, and multiple delivery catheters. Components may be exchanged via over-the-wire, rapid exchange, combination, or other approaches.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments. For example, although this disclosure has been written to describe devices that are generally described as being used to create a path of fluid communication between the left atrium and the right atrium, it should be appreciated that similar embodiments could be utilized for shunts between other chambers of the heart or for shunts in other regions of the body.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A shunting system for fluidly connecting a first body region and a second body region of a patient, the system comprising:
an anchor structure configured to stabilize the shunting system across a target anatomical structure; and
an actuation assembly coupled to the anchor structure, wherein the actuation assembly includes—
an actuator composed of one or more shape memory wires, the actuator including a plurality of projections that together define a cylindrical or conical shape having a lumen extending therethrough, wherein tips of the projections define an opening to the lumen, and
a membrane covering the plurality of projections, wherein the membrane individually covers individual projections of the plurality of projections,
wherein the actuation assembly is selectively transitionable between at least two or more configurations, and wherein the at least two or more configurations are associated with different fluid resistances through the lumen.

2. The system of claim 1 wherein the at least two or more configurations include a reset position and one or more expanded configurations.

3. The system of claim 2 wherein:
when the actuation assembly is in the reset position, the lumen has a first resistance, and
when the actuation assembly is in one of the one or more expanded configurations, the lumen has a second resistance,
wherein the first resistance is greater than the second resistance.

4. The system of claim 2 wherein:
when the actuation assembly is in the reset position, the opening has a first diameter, and
when the actuation assembly is in one of the one or more expanded configurations, the opening has a second diameter,
wherein the first diameter is less than the second diameter.

5. The system of claim 2 wherein:
the actuator is configured to transition between (a) a first material state having relatively less stiff mechanical properties and (b) a second material state having relatively stiffer mechanical properties in response to being heated above a transition temperature that is greater than body temperature, and
the actuation assembly is configured to return to the reset position in response to being heated above the transition temperature.

6. The system of claim 2 wherein, when the actuation assembly is in the reset position, neighboring projections of the plurality of projections at least partially overlap.

7. The system of claim 6 wherein neighboring projections overlap by between about 0.1 mm and about 2.5 mm.

8. The system of claim 6 wherein neighboring projections overlap by between about 0.5 mm and about 1.5 mm.

9. The system of claim 2 wherein neighboring projections of the plurality of projections at least partially overlap in both the reset position and the one or more expanded positions.

10. The system of claim 1 wherein neighboring projections of the plurality of projections are separated by a gap.

11. The system of claim 10 wherein the gap is at least about 0.5 mm.

12. The system of claim 10 wherein the gap is at least about 1 mm.

13. The system of claim 1 wherein the membrane includes a plurality of individual membranes, and wherein individual membranes of the plurality of membranes jacket individual projections of the plurality of projections.

14. The system of claim 13 wherein the individual membranes each include a first membrane portion and a second membrane portion divided at a waist, and wherein the first membrane portion jackets an individual projection and the second membrane portion extends over a portion of the anchor structure.

15. The system of claim 14 wherein the first membrane portion includes two layers and the second membrane portion includes a single layer.

16. The system of claim 14 wherein the first membrane portion includes two layers and the second membrane portion includes two layers.

17. The system of claim 1 wherein the membrane includes a single membrane having a plurality of membrane portions separated by gaps, and wherein individual membrane portions of the plurality of membrane portions cover individual projections of the plurality of projections.

18. The system of claim 1 wherein the membrane is composed of ePTFE.

19. The system of claim 1 wherein the adjustable shunting system is an interatrial shunting system, and wherein the target anatomical structure is a septal wall of a heart between a left atrium and a right atrium.

20. The system of claim 1 wherein the membrane is a first membrane, and wherein the system further comprises a second membrane covering the anchor structure.

21. The system of claim 20 wherein the first membrane and the second membrane are discontinuous.

22. The system of claim 20 wherein the second membrane is composed of a biocompatible and/or anti-thrombogenic material.

23. The system of claim 1 wherein the anchor structure is electrically coupled to the actuator via a capacitor.

24. The system of claim 23 wherein the anchor structure, the actuator, and the capacitor form an RLC circuit.

25. A shunting system for fluidly connecting a first body region and a second body region of a patient, the system comprising:
a plurality of petals each extending between a first end portion and a second end portion, wherein the petals are arranged in a cylindrical or conical shape to form a lumen therethrough, with an opening to the lumen defined by the first end portions of the petals;
wherein each individual petal of the plurality of petals includes a corresponding individual membrane or individual membrane portion such that at least the first end portions of neighboring petals can move relative to one another without stretching the individual membranes or individual membrane portions.

26. The system of claim 25 wherein the petals are configured such that the petals can flex radially inwardly and/or radially outwardly without stretching the membrane or membrane portions.

27. The system of claim 26 wherein the opening size is adjustable by flexing the petals radially inwardly and/or radially outwardly.

28. The system of claim 25 wherein the membrane or membrane portion is stiff.

29. The system of claim 25 wherein neighboring petals of the plurality of petals overlap.

30. The system of claim 25 wherein neighboring petals of the plurality of petals are spaced apart by a gap.

31. The system of claim 25 wherein the plurality of petals include a shape memory wire extending therethrough.

32. An adjustable shunting system for fluidly connecting a first body region and a second body region of a patient, the system comprising:
a plurality of membranes arranged in a conical or cylindrical shape to form a lumen therethrough,
wherein each membrane has a perimeter including a first edge extending at least partially along a length of the lumen, a second edge extending at least partially along the length of the lumen, and a tip portion connecting the first edge and the second edge, wherein a width between the first edge and the second edge is less than a circumference of the lumen.

33. The system of claim 32 wherein the width between the first edge and the second edge varies along a length of each membrane.

34. The system of claim 32 wherein neighboring membranes of the plurality of membranes at least partially overlap.

35. The system of claim 32 wherein neighboring membranes of the plurality of membranes are separated by a gap.
